(19) United States
(12) Patent Application Publication
(10) Pub. No.: US 2026/0069281 A1
(43) Pub. Date: Mar. 12, 2026

(54) BRAIDED IMPLANTABLE ENDOVASCULAR EMBOLIZATION DEVICE FREELY ROTATABLE RELATIVE TO A PUSHING MEMBER DURING ADVANCEMENT IN A MICROCATHETER

(71) Applicant: DePuy Synthes Products, Inc., Raynham, MA (US)

(72) Inventors: Daniel SOLAUN, Miami, FL (US); Pedro D. PEDROSO, Parkland, FL (US); Lacey GOROCHOW, Miami, FL (US)

(73) Assignee: DePuy Synthes Products, Inc., Raynham, MA (US)

(21) Appl. No.: 18/828,615

(22) Filed: Sep. 9, 2024

(51) Int. Cl. A61B 17/12; A61B 17/00

(52) U.S. Cl. CPC A61B 17/12172; A61B 17/12113; A61B 2017/00292; A61B 2017/00358; A61B 2017/12063

(57) ABSTRACT

An endovascular embolization system including a braided implantable device having: a braided structure radially self-expanding and having a proximal edge; a proximal securement mechanism fixedly secured radially constraining a proximal section of the braided structure including the proximal edge; the proximal section of the braided structure defining an inner channel having an inner diameter smaller relative to an interior space disposed distally of the inner channel; a pushing member having a proximal end and a distal end; no portion of the pushing member being rotatable relative to another portion; and an unrestricted 360 degrees freely rotatable connection of the braided implantable device relative to the pushing member thereby preventing or minimizing longitudinal twisting of the braided implantable device during delivery through a microcatheter.

### FIELD

The present disclosure relates to an endovascular medical treatment device, and more particularly, to a braided implantable endovascular embolization device to disrupt blood flow at the target site. By way of non-limiting example, the braided implantable endovascular embolization device may be used in the treatment of an aneurysm to divert (i.e., impede) blood flow or to restrict blood flow in a vessel (e.g., in the brain or peripheral vasculature). The braided implantable endovascular embolization device is unrestricted 360 degrees freely rotatable relative to a pushing member of a delivery system. Thus, during delivery via the microcatheter through the tortuous vasculature to the target site the braided implantable endovascular embolization device automatically or freely self-rotates (i.e., untwists) about a longitudinal axis in response to any imposed torque friction force.

### BACKGROUND

An aneurysm stretches out thereby thinning a section of the wall of the artery. Cranial aneurysms may be difficult to treat due to their proximity to critical brain tissues. Conventional solutions have included endovascular treatment whereby an internal volume of the aneurysm sac is surgically removed or excluded from arterial blood pressure and flow via an endovascular intrasaccular device. Current alternatives to endovascular intrasaccular devices or other surgical approaches include endovascularly delivered treatment devices that fill the sac (i.e., dome) of the aneurysm with embolic material or block the entrance (i.e., neck) of the aneurysm. Both approaches attempt to prevent or divert blood flow into the aneurysm. By filling an aneurysm sac, the embolic material clots the blood, creating a thrombotic mass within the aneurysm. Whereas, treating the aneurysm neck, blood flow into the entrance of the aneurysm is impeded, inducing venous stasis in the aneurysm and facilitating a natural formation of a thrombotic mass within the aneurysm.

Current intravascularly delivered implantable embolization devices typically utilize multiple devices (e.g., embolic coils) to either fill the sac or treat the entrance (i.e., neck) of the aneurysm. Naturally formed thrombotic masses created by treating the entrance with embolic coils may result in improved healing compared to aneurysm masses packed with embolic coils because naturally formed thrombotic masses may reduce the likelihood of distention from arterial walls and facilitate reintegration into the original parent vessel shape along the neck plane. However, embolic coils delivered to the neck of the aneurysm may potentially have the adverse effect of impeding the flow of blood in the adjoining blood vessel, particularly if the entrance is overpacked. Conversely, if the entrance is insufficiently packed, recanalization of blood flow may persist into the aneurysm. Treating certain aneurysm morphology (e.g., wide neck, bifurcation, etc.) may require ancillary devices (e.g., stents or balloons) to support the coil mass and obtain the desired packing density. Once implanted, the coils are not easily retractable or repositionable. Furthermore, aneurysms treated with multiple coils over time often recanalize or compact resulting from poor coiling, lack of coverage across the aneurysm neck, blood flow, and/or relatively large aneurysm size.

Alternatives to embolic coils are being explored, for example a tubular braided implant as disclosed in U.S. Pat. Nos. 10,653,425; 10,751,066; 11,278,292; 11,413,046; and 11,583,282, each of which is incorporated herein by reference in their entirety. Tubular braided implants have the potential to easily, accurately, and safely treat an aneurysm or other arterio-venous malformation in a parent vessel without blocking flow into perforator vessels communicating with the parent vessel. Compared to embolic coils, however, tubular braided implants are a newer technology, and there is therefore capacity for improved geometries, configurations, delivery systems, optimization of disruption of blood flow, etc. Regarding the geometry, it is desirable to design the tubular braided implant to minimize risk of damage to the vessel wall. This is a factor in all vasculature treatment procedures, but particularly significant during treatment of an aneurysm in which the vessel wall is inherently thin. Several factors contribute to optimizing disruption of blood flow. During delivery through the microcatheter, the conventional braided implantable device may undesirably twist in configuration relative to the axial/longitudinal axis extending therethrough hampering maximum radial expansion during deployment and implantation at the target site. Moreover, the efficiency of the conventional braided implantable device to disrupt blood flow to the aneurysm is compromised when twisted. Despite most likely being unsuccessful attempts may be made to untwist the conventional braided implantable device by torquing the delivery wire once the implant has exited from the distal end of the microcatheter (i.e., once deployed or implanted at the target site in the vessel of the patient). If not successfully untwisted once deployed or implanted, then the twisted conventional braided implantable device must be fully withdrawn from the microcatheter. Once outside the body, the twisted conventional braided implantable device may be manipulated by hand to its original untwisted orientation prior to reattempting delivery of the same device. Otherwise following withdraw of the twisted conventional braided implantable device from the body a new braided implantable device may be delivered through the microcatheter to the target site. Efficiency of diversion of blood flow may also be optimized by preventing or minimizing probability of distal migration over time of the tubular braided implanted device (i.e., future recanalization).

It is therefore desirable to develop an improved braided implantable endovascular embolization device that throughout the procedure (including during delivery through the microcatheter and upon exiting from the distal end thereof) prevents or minimizes undesirable twisting.

### SUMMARY

An aspect of the present disclosure relates to an improved braided implantable endovascular embolization device that at all times, and in particular, including during delivery through the delivery device (e.g., microcatheter, catheter, guide catheter, or delivery catheter) to the target site and subsequent deployment at the target site, is preferably unrestricted 360 degrees freely rotatable relative to the pushing member comprising part of the delivery system minimizing or preventing twisting about a longitudinal axis thereby ensuring maximum radial expansion and optimizing efficiency of diversion of blood flow when implanted at the target site in its natural, default, or original (i.e., non-twisted) configuration.

Another aspect of the present disclosure relates to an improved braided implantable endovascular embolization device unrestricted 360 degrees freely rotatable relative to the pushing member preventing twisting about the longitudinal axis during delivery through the microcatheter and resulting built-up energy therefore decreasing the track force allowing delivery via smaller size microcatheters.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices of the present disclosure, by way of example only, not by way of limitation.

FIG. 1A is a side view of an example freely rotatable connection of a generic braided implantable endovascular embolization device relative to a pushing member; the generic braided implantable endovascular embolization device is unrestricted 360 degrees freely rotatable about a tether having a distal interference member disposed at the distal end; illustrated in an attached state prior to electrolytic detachment of the tether from the pushing member; the illustration depicting cut away a partial cut away view of the generic braided implantable endovascular embolization device as well as the proximal marker band;

FIG. 1B depicts the generic braided implantable endovascular embolization device of FIG. 1A in a detached state following electrolytic severing of the tether proximate the interface with the pushing member; the illustration depicting a partial cut away view of the generic braided implantable endovascular embolization device;

FIG. 1C depicts an exemplary configuration of the electrolytic detachment components of the delivery system in FIG. 1A including a conductive metal wire extending longitudinally/axially through the pushing member, the tether, and into a portion of the distal interference member of FIG. 1A; illustrating the attached state with the proximal end of the tether secured (i.e., non-severed) to the distal end of the pushing member; the illustration depicting a partial longitudinal cross-sectional view of a proximal section of the pushing member and tether prior to electrolytic detachment;

FIG. 1D depicts the exemplary configuration of the electrolytic detachment of FIG. 1C in a detached state with the proximal end of the tether electrolytically severed from the distal end of the pushing member; the illustration depicting a partial longitudinal cross-sectional view of a proximal section of the pushing member after electrolytic detachment from the tether;

FIG. 2A is a side view of another example freely rotatable connection of a generic braided implantable endovascular embolization device relative to a pushing member; the generic braided implantable endovascular embolization device is unrestricted 360 degrees freely rotatable about a tether and movement of the tether in a longitudinal/axial direction restricted by a distal interference member and a proximal interference member; illustrated in an attached state prior to electrolytic detachment of the tether from the pushing member; the illustration depicting partial cut away view of the generic braided implantable endovascular embolization device as well as the proximal marker band;

FIG. 2B depicts the generic braided implantable endovascular embolization device of FIG. 2A in a detached state electrolytically severed at the interface of the proximal interference member and the distal end of the pushing member; in the detached state distal migration of the distal interference member into the interior space of the braided implantable endovascular embolization device is curtailed by the proximal interference member; the illustration depicting a partial cut away view of the generic braided implantable endovascular embolization device;

FIG. 2C depicts an exemplary configuration of the electrolytic detachment components of the delivery system in FIG. 2A including a conductive metal wire extending longitudinally/axially through the pushing member, the proximal interference member, the tether, and into a portion of the distal interference member of FIG. 2A; illustrating the state with the proximal interference member attached to the distal end of the pushing member; the illustration depicting a partial longitudinal cross-sectional view of a proximal section of the pushing member and tether prior to electrolytic detachment;

FIG. 2D depicts the exemplary configuration of the electrolytic detachment components of FIG. 2C in a state with the proximal interference member detached (i.e., electrolytically severed) from the distal end of the pushing member; the illustration depicting a partial longitudinal cross-sectional view of a proximal section of the pushing member after electrolytic detachment from the tether;

FIG. 3A depicts still another exemplary freely rotatable connection of a generic braided implantable endovascular embolization device relative to a delivery wire; the generically depicted braided implantable endovascular embolization device is unrestricted 360 degrees freely rotatable about a tether and movement of the tether in a longitudinal/axial direction is restricted by a distal interference member and a detachment member; illustrated in a secured state prior to mechanical detachment/release (via the pull wire) of the tether from the pushing member; the illustration depicting a partial cut away view of the generic braided implantable endovascular embolization device as well as the proximal marker band;

FIG. 3B depicts the generic braided implantable endovascular embolization device of FIG. 3A following release (e.g., mechanical detachment) of the detachment member from the pushing member via the pull wire in a retracted (i.e., unsecured or free) state; the illustration depicting a partial cut away view of the generic braided implantable endovascular embolization device;

FIG. 4 is a flow chart of operation of the generic braided implantable endovascular embolization device unrestricted 360 degrees freely rotatably connected relative to the pushing member in accordance with the present disclosure; and

FIG. 5 is a flow chart of method of manufacture of the generic braided implantable endovascular embolization device unrestricted 360 degrees freely rotatably connected relative to the pushing member in accordance with the present disclosure.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

One aspect of the present disclosure is directed to a single device for diversion/disruption of blood flow, e.g., diversion of blood flow to an aneurysm or impeding blood flow in a vessel such as in the brain or peripheral vasculature. During treatment of an aneurysm, rather than employing the time-consuming process of packing the sac/dome of the aneurysm with multiple embolization devices (e.g., multiple conventional embolic coils typically of varying size), the delivery and deployment procedure in accordance with the present disclosure is streamlined to only a single assembled braided implantable endovascular embolization device. The focus of the present disclosure is the unrestricted 360 degrees freely rotatable connection of the braided implantable endovascular embolization device relative to the pushing member of the delivery system. This feature is applicable to any generic braided implantable endovascular embolization device (i.e., hence use of the term “generic” to convey that the unrestricted 360 degrees freely rotatable connection feature relative to the pushing wire of the delivery system is applicable to any braided implantable endovascular embolization device irrespective of the configuration. Throughout the figures, for illustration purposes only, the braided implantable endovascular embolization device is depicted by a generic radially self-expanding structure (e.g., resembling that of an inflated balloon while in a pre-formed default state) having a closed distal end with a proximal marker band 110a secured about an opposite open proximal section terminating in a proximal edge. The configuration of the braided implantable endovascular embolization device is not critical to the present disclosure, the aspect of the present disclosure being the unrestricted 360 degrees freely rotatable connection of any braided implantable endovascular embolization device relative to the pushing member forming part of the delivery system. The braided implant is preferably made of a shape memory material (e.g., a polymer or metal such as Nitinol) in any desired configuration. In addition, the braid pattern (e.g., diamond), number of wires, thickness or diameter of each wire, shape of each wire, pic count, pitch, and any other parameter associated with the braided implant may be varied, as desired. Not only may the unrestricted 360 degrees freely rotatable connection aspect of the present disclosure be used with any configuration of braided implant but is also applicable to any delivery system employing a pushing member to advance the implant through the microcatheter to the target site.

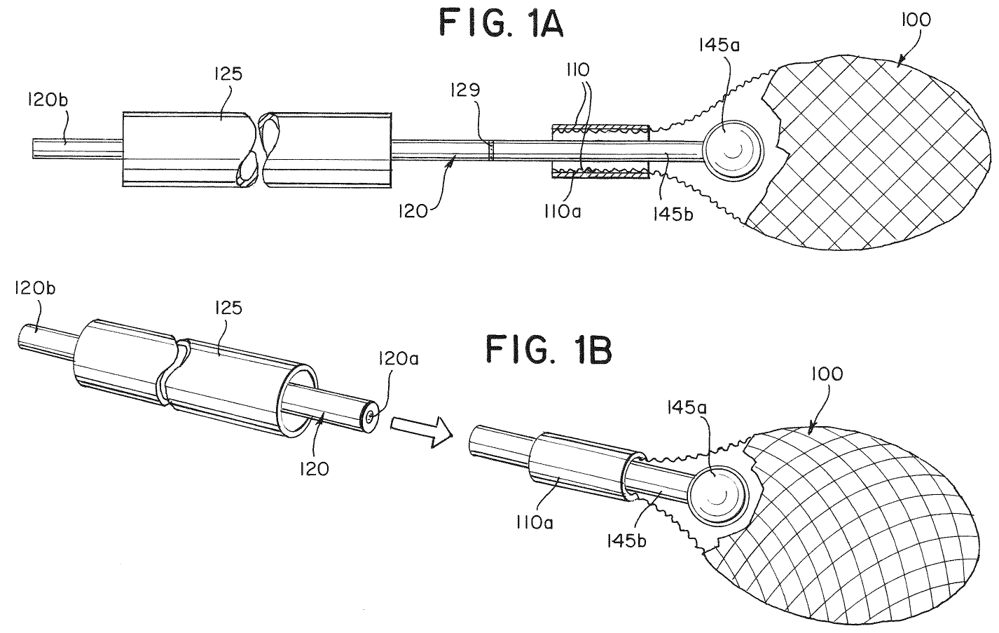

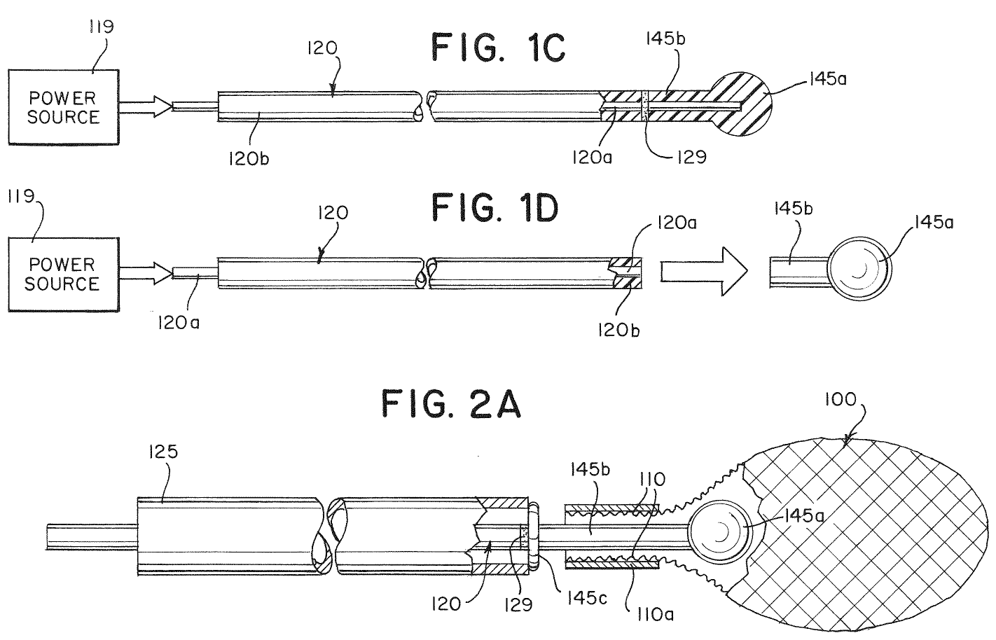

An example unrestricted 360 degrees freely rotatable connection of the generic braided implantable endovascular embolization device 100 relative to a pushing member 120 comprising part of a delivery system is shown in the side view of FIGS. 1A & 1B. The generic braided implantable endovascular embolization device 100 throughout the figures is formed of a plurality of wires woven into a desired pattern (e.g., full diamond pattern). To maximize compressibility and minimize risk of damage to the vessel wall each wire has an outer diameter preferably

≤approximately 0.002″. The wires are preferably made of a biocompatible memory shape material (e.g., polymer or metal such as Nitinol—Nickle titanium alloy) pre-formable (e.g., heat set) to have a natural, default, or original shape (i.e., radially self-expanded state of enlarged outer diameter) while free from an externally applied radially compressive or radially constraining force, but transitioning to a radially collapsed state having a reduced outer diameter when subject to the externally applied radially compressive or radially constraining force. In the natural, default, or original shape the generic braided implantable endovascular embolization device is untwisted (i.e., free from twisting) in a longitudinal/axial direction. Any configuration of the generic braided implantable endovascular embolization device may be employed. A proximal marker band 110a disposed about a proximal section 110 of the generic braided implantable endovascular embolization device including a proximal free edge is cinched in place (e.g., physically crimped or deformed) radially constraining the proximal section 110 to have an inner channel of reduced diameter relative to an interior space disposed distally thereof. Preferably, the outer diameter of the proximal section 110 with the proximal marker band 110a secured thereabout is advanceable through the microcatheter without requiring radial constriction and hence upon emerging from the distal end of the microcatheter does not experience radial expansion.

The unrestricted 360 degrees freely rotatable connection illustrated in the example of FIGS. 1A & 1B includes a tether 145b (e.g., wire) made of a biocompatible material (e.g., metal or polymer) extending longitudinally/axially through the inner channel of the proximal section 110 of the braided implantable endovascular embolization device 100 with the proximal marker band 110a physically secured (e.g., physically crimped or deformed) thereabout. Tether 145b has an outer diameter smaller than an inner diameter of the inner channel of the proximal section 110 provided sufficient radial clearance to allow unrestricted 360 degrees free rotation of the braided implantable endovascular embolization device 100 about the tether 145b therein. Permanently (i.e., fixedly, or non-releasably) secured to a distal end of the tether 145b is a distal interference member 145a disposed in the interior space or cavity of the braided implantable endovascular embolization device 100. The distal interference member 145a (e.g., a ball or other preferably atraumatic shape element) has an outer diameter larger than the inner diameter of the inner channel of the proximal section 110 preventing passage therethrough in a proximal direction. In the example unrestricted 360 degrees freely rotatable connection mechanism of FIGS. 1A & 1B, the tether 145b is permanently (i.e., fixedly or non-releasably) secured at its proximal end to the distal end of the pushing member 120 (e.g., delivery wire). After exiting from the distal end of the microcatheter 125 and implanted at the target site, the braided implantable endovascular embolization device 100 is detached from the pushing member 120 by severing (e.g., electrolytic) the tether 145b proximally of the distal interference member 145b, preferably at the interface of the proximal end of the tether 145b and the distal end of the pushing member 120 (FIG. 1B). An exemplary electrolytic detachment system associated with the pushing member 120 and tether 145b of FIGS. 1A & 1B is shown in FIG. 1C (prior to severing/detachment) and FIG. 1D (after severing/detachment). The electrolytic detachment system includes an electrical wire 120a extending at least to the distal end/tip of the pushing member 120. Optionally, the electrical wire 120a may extend still further in a distal direction into at least a portion of the tether 145b and/or into a portion of the distal interference member 145a. An outer jacket or layer 120b made of an electrically insulating material covers (i.e., insulates) the electrical wire 120a everywhere except for a non-insulated detachment region 129. In the example of FIGS. 1C & 1D the non-insulated detachment region 129 is disposed proximally of the distal interference member 145a, preferably at the interface of the proximal end of the tether 145b and the distal end of the pushing member 120. An electrical current generated by a power source 119 (e.g., battery) is applied to the conductive metal wire 120a causing its erosion only in the non-insulated detachment region 129 resulting in detachment (i.e., severing or separation) of the tether 145b from the pushing member 120 and release of the braided implantable endovascular embolization device 100 from the delivery system (e.g., pushing member 120). Following detachment, the implanted braided implantable endovascular embolization device 100 together with the distal interference member 145a and severed portion of the tether 145b attached thereto remains implanted at the target site while the delivery system (e.g., pushing member 120 and microcatheter 125) is withdrawn (e.g., serially one after the other or simultaneously together) from the body.

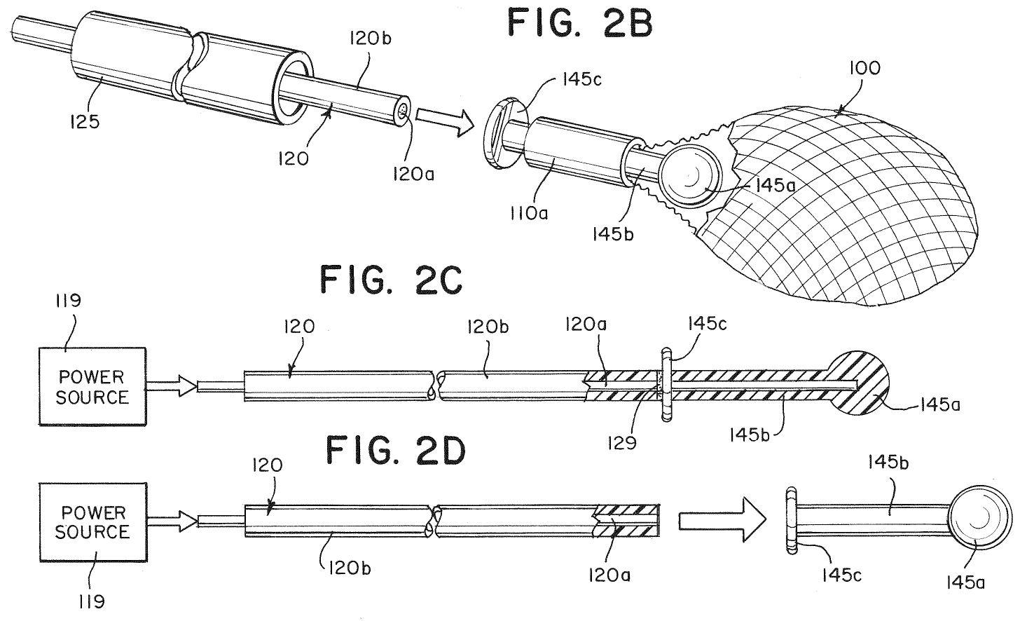

Another example unrestricted 360 degrees freely rotatable connection of the braided implantable endovascular embolization device 100 relative to the pushing member 120 (e.g., delivery wire) is depicted in FIGS. 2A & 2B. The unrestricted 360 degrees freely rotatable connection of FIGS. 2A & 2B differs from that of FIGS. 1A & 1B with the inclusion of a proximal interference member 145c fixedly secured to the proximal end of the tether 145b and fixedly (i.e., permanently or non-releasably) secured to the distal end of the pushing member 120. Once implanted at the target site, the braided implantable endovascular embolization device 100 is detached from the delivery system (e.g., pushing member 120) by severing (e.g., electrolytic) the tether 145b at the interface of the proximal interference member 145c and the distal end of the pushing member 120 (FIG. 2B). An exemplary electrolytic detachment system associated with the pushing member 120 (e.g., delivery wire) and tether 145b of FIGS. 2A & 2B is shown in FIG. 2C (prior to severing/detachment) and FIG. 2D (after severing/detachment). In the example electrolytic detachment system of FIGS. 2C & 2D, the pushing member 120 comprises a conductive metal wire 120a extending at least to the distal end/tip of the pushing member 120. Optionally, the conductive metal wire 120a may extend further in a distal direction into at least a portion of the proximal interference member 145c, the tether 145b and/or into the distal interference member 145a. An outer jacket or layer 120b made of an electrically insulating material covers (i.e., insulates) the conductive metal wire 120a everywhere except for a non-insulated detachment region 129. In the example of FIGS. 2C & 2D the non-insulated detachment region 129 is disposed at the interface of the proximal interference member 145c and the distal end of the pushing member 120. An electrical current generated by the power source 119 (e.g., battery) is applied to the conductive metal wire 120a causing erosion only in the non-insulated detachment region 129 resulting in severing/detachment of the tether 145b from the pushing member 120 and release of the braided implantable endovascular embolization device 100 from the delivery system (e.g., pushing member 120). Following detachment, the microcatheter 125 including the pushing member 120 is withdrawn from the body (e.g., serially one after the other or simultaneously together) while the deployed braided implantable endovascular embolization device 100 together with the distal interference member 145a, the tether 145b, and the proximal interference member 145c remains implanted at the target site. In this example depicted in FIGS. 2A-2D, longitudinal movement of the detached tether 145b in the inner channel of the proximal section 110 is restricted by the respective distal and proximal interference members 145a, 145c, respectively, on opposite ends of the tether 145b.

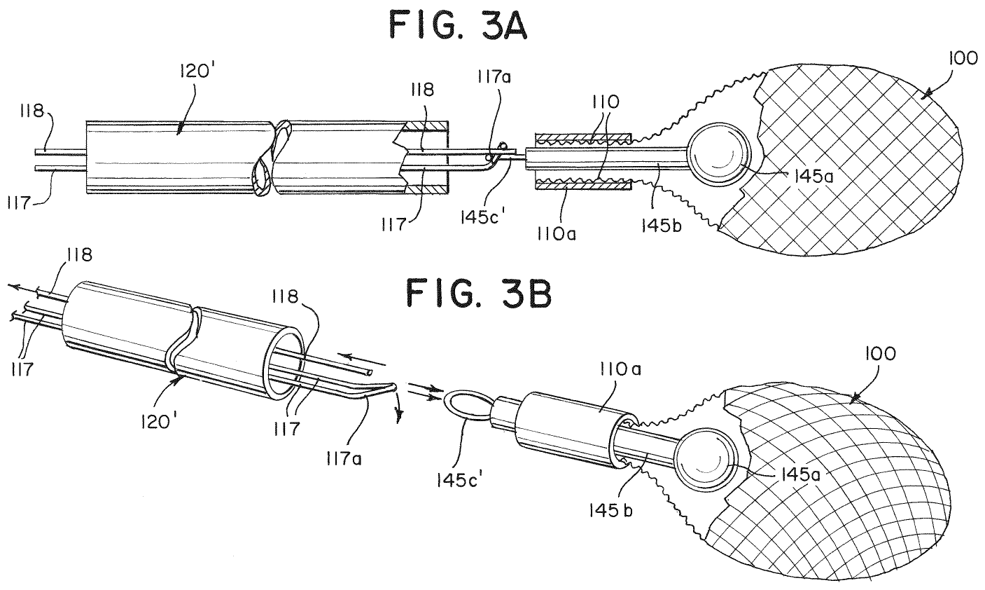

Yet another example unrestricted 360 degrees freely rotatable connection of the braided implantable endovascular embolization device 100 relative to the pushing member 120 (e.g., delivery tube) is shown in FIGS. 3A & 3B. This alternative example uses mechanical detachment of the braided implantable endovascular embolization device 100 from the pushing member 120, in contrast to the electrolytic detachment in FIGS. 1A-1D & 2A-2D described above. Referring to the example in FIGS. 3A & 3B, the unrestricted 360 degrees freely rotatable connection includes a tether 145b having a distal interference member 145a fixedly (i.e., permanently or non-releasably) mounted to the distal end. Fixedly (i.e., permanently or non-releasably) mounted to the opposite proximal end of the tether 145b is a detachment member 145c′ (i.e., key). In the example depicted in FIGS. 3A & 3B, the key 145c′ is an eyelet or closed circular loop, but any other closed loop shape having an opening defined therein is also possible. Detachment member 145c′ has a maximum outer diameter larger than the diameter of the inner channel of the proximal section 110 of the braided implantable endovascular embolization device 100 preventing passage therethrough serving a dual function as a securement feature to releasably attach the two components and as a proximal interference member restricting distal migration of the tether 145b following detachment. The pushing member 120′ (e.g., delivery tube) has defined therein in a longitudinal/axial direction an inner lumen from the distal end to the opposite proximal end. Extending longitudinally/axially through the inner lumen and protruding out from the respective proximal and distal ends of the tube 120′ is a release or pull wire 118. That portion of the pull wire 118 protruding in a proximal direction from the proximal edge of the pushing member 120′ serves as a handle that when grasped and pulled in the proximal direction via a physician or interventionalist releases (i.e., frees or detaches) the braided implantable endovascular embolization device 100 from the pushing member 120'. Separate from the pull wire 118, a securement wire 117 folded over itself, preferably in half, forms a loop 117a at a distal end with the opposite proximal ends extending in a proximal direction. The proximal ends of the securement wire 117 may be permanently secured (e.g., adhered or welded) interiorly or exteriorly to the tube 120'. Otherwise, the proximal ends of the securement wire 117 may extend in a proximal direction completely through the lumen of the tube 120 and held in place manually by the physician or interventionalist. During attachment or assembly of the braided implantable endovascular embolization device 100 to the pushing member 120′ the loop 117a at the distal end of the securement wire 117 is passed through the opening defined in the detachment member 145c′ (i.e., key)(e.g., closed circular loop) mounted to the proximal end of the tether 145b. Then, the distal end of the pull wire 118 is positioned through the loop 117a of the securement wire 117 while threaded through the detachment member 145c′ (FIG. 3A). Thus, the braided implantable endovascular embolization device 100 and the delivery system 120′ are attached together via the pull wire 118. The physician or interventionalist pulls in a proximal direction on the projecting free proximal end of the pull wire 118 until its opposite distal end is freed from the loop 117a of the securement wire 117 thereby releasing the two components from one another (FIG. 3B). Following mechanical detachment (i.e., release), the pushing member 120′ along with the securement wire 117 and the pull wire 118 are withdrawn from the body while the deployed braided implantable endovascular embolization device 100 together with the detachment member 145c′, the tether 145b, and the distal interference member 145a remains implanted at the target site.

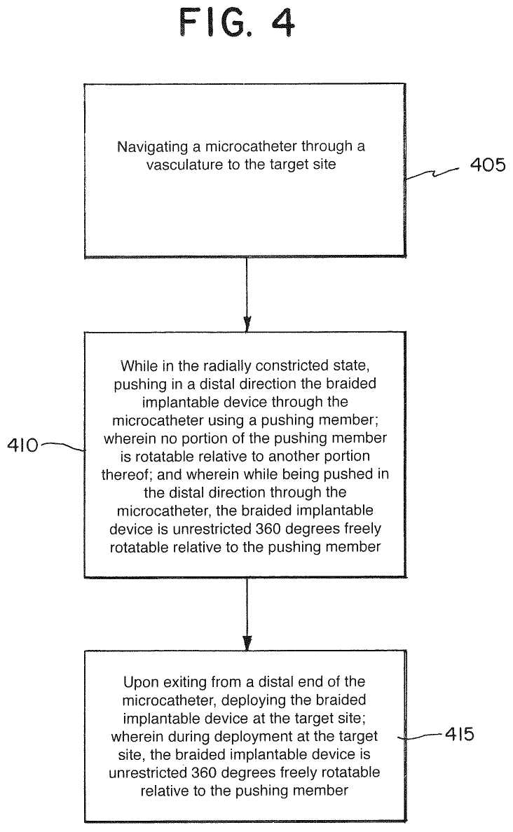

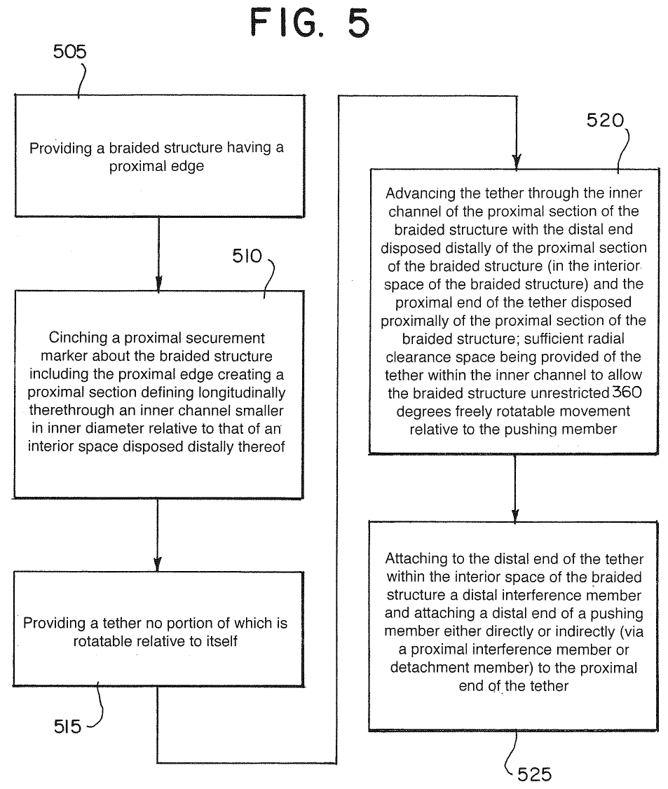

FIG. 4 is a flow chart of use of the unrestricted 360 degrees freely rotatable connection of the braided implantable endovascular embolization device 100 relative to the delivery device (e.g., pushing member 120, 120′) in the treatment at a target site (e.g., an aneurysm or the occlusion of blood flow in a vessel). Initially, in step 405 the microcatheter 125 is navigated through a vasculature to the target site. For example, a guide wire and a delivery catheter, either independently in series one after the other or simultaneously together, may navigate through the vasculature to the target site. Once positioned at the target site, the guide wire is withdrawn proximally from the body followed by the subsequent advancement through the delivery wire of a microcatheter 125 to the target site to be treated. Thereafter, in step 410, while in the radially constricted state, the braided implantable endovascular embolization device 100 is advanced (i.e., pushed in a distal direction) through the microcatheter 125 using a pushing member 120, 120′ (e.g., delivery wire or tube) of a delivery system. No portion of the pushing member 120, 120′ is rotatable relative to another portion of the pushing member. While advanced through the microcatheter 125 the braided implantable endovascular embolization device 100 is unrestricted 360 degrees freely rotatable relative to the pushing member 120, 120′. Specifically, the braided implantable endovascular embolization device 100 is unrestricted 360 degrees freely rotatable about the tether 145b. Accordingly, any potential twisting of the braided implantable endovascular embolization device 100 about the longitudinal axis defined therethrough resulting from torque friction while navigating through the lumen of the microcatheter 125 of the tortuous vasculature is automatically self-untwisted (i.e., reverting to a fully untwisted state) via the unrestricted 360 degrees freely rotatable connection of the two components. Upon exiting or emerging from the distal end of the microcatheter 125 in step 415, the braided implantable endovascular embolization device 100 self-expands radially to an implanted (i.e., deployed) state at the target site (e.g., aneurysm or blood vessel). Any residual, minimum, or de minimis twisting of the braided implantable endovascular embolization device 100 not automatically self-untwisted during delivery through the microcatheter 125 upon exiting therefrom automatically freely rotates (i.e., self-untwist) via the unrestricted 360 degrees freely rotatable connection reverting to its original, natural, default, fully untwisted state. Thus, any undesirable twisting of the braided implantable endovascular embolization device experienced during delivery through the microcatheter to the target site is compensated (i.e., self-untwisting or automatically reverting to an original, natural, default fully untwisted state) during advancement (i.e., delivery) through or upon exiting from the microcatheter 125. Allowing unrestricted 360 degrees free rotation of the braided implantable endovascular embolization device 100 relative to the pushing member 120, 120′ in response to any twisting during delivery through the microcatheter 125 ensures that the device implanted at the target site is fully, completely, or totally untwisted thereby maximizing radial expansion of the deployed implant 100 at the target site and efficiency of the disruption of blood flow.

A flow chart of the method of manufacture of the braided implantable endovascular embolization device unrestricted 360 degrees freely rotatably connected to the delivery system (e.g., pushing member 120, 120′) in accordance with the present disclosure is provided in FIG. 5. In step 505, a braided structure is provided having a proximal edge. As previously mentioned, the pre-formed outer contour, profile, or shape of the braided structure while in the radially expanded state may be selected, as desired. Depicted in the figures by way of generic illustration only, the pre-formed shape of the braided structure while in a default, original, natural, radially expanded state resembles that of an inflated balloon. Then in step 510 a proximal securement marker 110 is radially cinched about the braided structure including the proximal edge creating a proximal section 110a defining longitudinally therethrough an inner channel of reduced inner diameter and an interior space of larger inner diameter disposed distally thereof. Next in step 515, a tether 145b is provided no portion of which is rotatable relative to any other portion of itself. In step 520, the tether 145b is advanced through the inner channel of the proximal section of the braided structure with the distal end disposed distally of the proximal section of the braided structure (in the interior space of the braided structure) and the proximal end of the tether 145b disposed proximally of the proximal section of the braided structure. Sufficient radial clearance space is provided of the tether 145b within the inner channel to allow the braided structure unrestricted 360 degrees freely rotatable movement relative to the pushing member 120. Lastly, in step 525, the distal interference member 145a is attached to the distal end of the tether 145b within the interior space of the braided structure while a distal end of the pushing member 120 is attached either directly or indirectly (e.g., via a proximal interference member 145c or detachment member 145c′) to the proximal end of the tether 145b.

Aspects of the present disclosure are also provided by the following numbered Clauses:
Clause 1: An endovascular embolization system comprising: a braided implantable device (100) comprising: a braided structure radially self-expanding and having a proximal edge; a proximal securement mechanism (110a) fixedly secured radially constraining a proximal section (110) of the braided structure including the proximal edge; the proximal section (110) of the braided structure defining an inner channel having an inner diameter smaller relative to an interior space disposed distally of the inner channel; a pushing member (120, 120′) having a proximal end and a distal end; no portion of the pushing member (120, 120′) being rotatable relative to another portion; and an unrestricted 360 degrees freely rotatable connection of the braided implantable device relative to the pushing member (120, 120′).
Clause 2: The system of Clause 1, wherein the unrestricted 360 degrees freely rotatable connection comprises a tether (145b) having a proximal end attached to the distal end of the pushing member (120, 120') and an opposite free distal end with a distal interference member (145a) fixedly secured thereto; the tether (145b) extending through the inner channel of the proximal section (110) of the braided structure with the distal interference member (145a) disposed in the interior space of the braided structure; wherein the tether (145b) disposed within the inner channel of the proximal section (110) of the braided structure defines a radial clearance space sufficient to allow unrestricted 360 degrees free rotation of the braided implantable device (100) about the tether (145b).
Clause 3: The system of Clause 2, wherein the tether (145b) is severable from the pushing member (120, 120′) via an electrolytic detachment mechanism comprising a conductive metal wire (120a) covered by an electrical insulating material (120b) everywhere except for a non-insulated detachment zone (129); and a power supply (119) electrically connected to the conductive metal wire (120a) for generating an electrical current therein.
Clause 4: The system of Clause 3, wherein the non-insulated detachment zone (129) is distally of the distal end of the pushing member (120).
Clause 5: The system of any of Clause 3 through 4, wherein the tether (145b) has at the proximal end a proximal interference member (145c) secured to the distal end of the of the pushing member (120); the proximal interference member (145c) restricting longitudinal movement in a distal direction of the tether (145b) through the inner channel of the proximal section (110) of the braided structure.
Clause 6: The system of Clause 5, wherein the proximal interference member (145c) is permanently secured to the distal end of the pushing member (120).
Clause 7: The system of Clause 5, wherein the non-insulated detachment zone is at an interface between the proximal interference member (145c) and the distal end of the pushing member (120).
Clause 8: The system of Clause 2, wherein the tether (145b) has at the proximal end a detachment member (145c') having an opening defined therein and releasably secured to the distal end of the pushing member (120′) via a mechanical detachment system.
Clause 9: The system of Clause 8, wherein the mechanical detachment system comprises: a securement wire (117) having a distal end with a loop (117a) projecting from the distal end of the pushing member (120′); and a release wire (118) transitionable between a secured state with the release wire (118) passing through the loop (117a) of the securement wire (117) and the opening of the detachment member (145c′) securing together the braided implantable device and the pushing member (120′) and a released state with the release wire (118) freed from the loop (117a) of the securement wire (117) and the opening of the detachment member (145c′) releasing the braided implantable device (100) from the pushing member (120').
Clause 10: A method for disrupting blood flow to a target site using an endovascular embolization system that includes a braided implantable device (100) comprising: a braided structure radially self-expanding and having a proximal edge; a proximal securement mechanism (110a) fixedly secured radially constraining a proximal section (110) of the braided structure including the proximal edge; the proximal section (110) of the braided structure defining an inner channel having an inner diameter smaller relative to an interior space disposed distally of the inner channel; a pushing member (120, 120′) having a proximal end and a distal end; no portion of the pushing member (120, 120′) being rotatable relative to another portion; and an unrestricted 360 degrees freely rotatable connection of the braided implantable device relative to the pushing member (120, 120′); the method comprising the steps of: navigating a microcatheter (125) through a vasculature to the target site; while in the radially constricted state, pushing in a distal direction the braided implantable device through the microcatheter (125) using the pushing member (120, 120′); and upon exiting from a distal end of the microcatheter (125), deploying the braided implantable device at the target site; wherein during the step of pushing through and/or exiting from the microcatheter (125) further comprising the step of the braided implantable device unrestricted 360 degrees freely rotating relative to the pushing member (120, 120') via the unrestricted 360 degrees freely rotatable connection.
Clause 11: The method of Clause 10, wherein the unrestricted 360 degrees freely rotatable connection comprises a tether (145b) having a proximal end attached to the distal end of the pushing member (120, 120′) and an opposite free distal end with a distal interference member (145a) fixedly secured thereto; the tether (145b) extending through the inner channel of the proximal section (110) of the braided structure with the distal interference member (145a) disposed in the interior space of the braided structure; wherein the tether (145b) disposed within the inner channel of the proximal section (110) of the braided structure defines a radial clearance space sufficient to allow the unrestricted 360 degrees free rotation of the braided implantable device (100) about the tether (145b).
Clause 12: The method of Clause 11, further comprising the step of severing the braided implantable device (100) from the pushing member (120) via an electrolytic detachment mechanism comprising a conductive metal wire (120a) covered by an electrical insulating material (120b) everywhere except for a non-insulated detachment zone (129); and a power supply (119) electrically connected to the conductive metal wire (120a) for generating an electrical current therein.
Clause 13: The method of Clause 12, wherein the non-insulated detachment zone (129) is distally of the distal end of the pushing member (120).
Clause 14: The method of Clause 12, wherein the tether (145b) has at the proximal end a proximal interference member (145c) secured to the distal end of the of the pushing member (120); the proximal interference member (145c) restricting longitudinal movement in a distal direction of the tether (145b) through the inner channel of the proximal section (110) of the braided structure.
Clause 15: The method of Clause 14, wherein the non-insulated detachment zone (129) is at an interface between the proximal interference member (145c) and the distal end of the pushing member (120).
Clause 16: The method of Clause 11, further comprising the step of releasing the braided implantable device (100) from the pushing member (120′) via a mechanical detachment system.
Clause 17: The method of Clause 16, wherein the tether (145b) has at the proximal end a detachment member (145c′) having an opening defined therein; and the mechanical detachment system comprises: a securement wire (117) having a distal end with a loop (117a) projecting from the distal end of the pushing member (120′); and a release wire (118) transitionable between a secured state with the release wire (118) passing through the loop (117a) of the securement wire (117) and the opening of the detachment member (145c′) securing together the braided implantable device and the pushing member (120′) and a released state with the release wire (118) freed from the loop (117a) of the securement wire (117) and the opening of the detachment member (145c′) releasing the braided implantable device (100) from the pushing member (120′).
Clause 18: A method for manufacture of an implantable endovascular embolization device (100), the method comprising the steps of: providing a braided structure having a proximal edge;
   cinching a proximal securement marker (110a) about the braided structure including the proximal edge creating a proximal section (110) defining longitudinally therethrough an inner channel having an inner diameter smaller relative to that of an interior space disposed distally thereof; providing a tether (145b) no portion of which is rotatable relative to itself; advancing the tether (145b) through the inner channel of the proximal section (110) of the braided structure with the distal end disposed distally of the proximal section of the braided structure in the interior space of the braided structure and the proximal end of the tether (145b) disposed proximally of the proximal section (110) of the braided structure; sufficient radial clearance space being provided of the tether (145b) within the inner channel to allow the braided structure unrestricted 360 degrees freely rotatable movement relative to the pushing member; attaching to the distal end of the tether (145b) within the interior space of the braided structure a distal interference member (145a) and attaching a distal end of a pushing member (120, 120') either directly or indirectly to the proximal end of the tether (145b).
Clause 19: The method of Clause 18, wherein the proximal end of the tether (145b) is indirectly permanently attached to the distal end of the pushing member (120′) via a proximal interference member (145c); wherein the proximal interference member (145c) restricts passage in a distal direction in the inner channel of the proximal section (110) of the braided structure.
Clause 20: The method of Clause 18, wherein the proximal end of the tether (145b) is indirectly releasably mechanically attached to the distal end of the pushing member (120′) via a detachment member (145c′); wherein the detachment member (145c′) restricts passage in a distal direction in the inner channel of the proximal section (110) of the braided structure.

The descriptions contained herein are examples and not intended in any way to limit the scope of the present disclosure. As described herein, the present disclosure contemplates many variations and modifications of the generic braided implantable endovascular embolization device unrestricted 360 degrees freely rotatably connected relative to a pushing member of a delivery system. Regardless of the particular configuration of the unrestricted 360 degrees freely rotatable connection, the generic braided implantable endovascular embolization device is not secured, mounted, connected or attached directly to the pushing member, rather it is unrestricted 360 degrees freely rotatable relative to the pushing member. It is the proximal securement member (e.g., collar, disc, ring or marker band) that is secured, mounted, connected or attached to the generic braided implantable endovascular embolization device, and the distal interference member disposed at the distal end of the tether prevents slippage/escape of the generic braided implantable endovascular embolization device off the pushing member. Modifications and variations apparent to those having skilled in the pertinent art according to the teachings of this disclosure regarding the detachment and delivery system are intended to be within the scope of the claims which follow.

What is claimed is:

1. An endovascular embolization system comprising:
a braided implantable device comprising:
a braided structure radially self-expanding and having a proximal edge;
a proximal securement mechanism fixedly secured radially constraining a proximal section of the braided structure including the proximal edge; the proximal section of the braided structure defining an inner channel having an inner diameter smaller relative to an interior space disposed distally of the inner channel;
a pushing member having a proximal end and a distal end; no portion of the pushing member being rotatable relative to another portion; and
an unrestricted 360 degrees freely rotatable connection of the braided implantable device relative to the pushing member.

2. The system in accordance with claim 1, wherein the unrestricted 360 degrees freely rotatable connection comprises a tether having a proximal end attached to the distal end of the pushing member and an opposite free distal end with a distal interference member fixedly secured thereto; the tether extending through the inner channel of the proximal section of the braided structure with the distal interference member disposed in the interior space of the braided structure; wherein the tether disposed within the inner channel of the proximal section of the braided structure defines a radial clearance space sufficient to allow unrestricted 360 degrees free rotation of the braided implantable device about the tether.

3. The system in accordance with claim 2, wherein the tether is severable from the pushing member via an electrolytic detachment mechanism comprising a conductive metal wire covered by an electrical insulating material everywhere except for a non-insulated detachment zone; and a power supply electrically connected to the conductive metal wire for generating an electrical current therein.

4. The system in accordance with claim 3, wherein the non-insulated detachment zone is distally of the distal end of the pushing member.

5. The system in accordance with claim 3, wherein the tether has at the proximal end a proximal interference member secured to the distal end of the of the pushing member; the proximal interference member restricting longitudinal movement in a distal direction of the tether through the inner channel of the proximal section of the braided structure.

6. The system in accordance with claim 5, wherein the proximal interference member is permanently secured to the distal end of the pushing member.

7. The system in accordance with claim 5, wherein the non-insulated detachment zone is at an interface between the proximal interference member and the distal end of the pushing member.

8. The system in accordance with claim 2, wherein the tether has at the proximal end a detachment member having an opening defined therein and releasably secured to the distal end of the pushing member via a mechanical detachment system.

9. The system in accordance with claim 8, wherein the mechanical detachment system comprises:
a securement wire having a distal end with a loop projecting from the distal end of the pushing member; and
a release wire transitionable between a secured state with the release wire passing through the loop of the securement wire and the opening of the detachment member securing together the braided implantable device and the pushing member and a released state with the release wire freed from the loop of the securement wire and the opening of the detachment member releasing the braided implantable device from the pushing member.

10. A method for disrupting blood flow to a target site using an endovascular embolization system that includes a braided implantable device comprising: a braided structure radially self-expanding and having a proximal edge; a proximal securement mechanism fixedly secured radially constraining a proximal section of the braided structure including the proximal edge; the proximal section of the braided structure defining an inner channel having an inner diameter smaller relative to an interior space disposed distally of the inner channel; a pushing member having a proximal end and a distal end; no portion of the pushing member being rotatable relative to another portion; and an unrestricted 360 degrees freely rotatable connection of the braided implantable device relative to the pushing member; the method comprising the steps of:
navigating a microcatheter through a vasculature to the target site;
while in the radially constricted state, pushing in a distal direction the braided implantable device through the microcatheter using the pushing member; and
upon exiting from a distal end of the microcatheter, deploying the braided implantable device at the target site;
wherein during the step of pushing through and/or exiting from the microcatheter further comprising the step of the braided implantable device unrestricted 360 degrees freely rotating relative to the pushing member via the unrestricted 360 degrees freely rotatable connection.

11. The method in accordance with claim 10, wherein the unrestricted 360 degrees freely rotatable connection comprises a tether having a proximal end attached to the distal end of the pushing member and an opposite free distal end with a distal interference member fixedly secured thereto; the tether extending through the inner channel of the proximal section of the braided structure with the distal interference member disposed in the interior space of the braided structure; wherein the tether disposed within the inner channel of the proximal section of the braided structure defines a radial clearance space sufficient to allow the unrestricted 360 degrees free rotation of the braided implantable device about the tether.

12. The method in accordance with claim 11, further comprising the step of severing the braided implantable device from the pushing member via an electrolytic detachment mechanism comprising a conductive metal wire covered by an electrical insulating material everywhere except for a non-insulated detachment zone; and a power supply electrically connected to the conductive metal wire for generating an electrical current therein.

13. The method in accordance with claim 12, wherein the non-insulated detachment zone is distally of the distal end of the pushing member.

14. The method in accordance with claim 12, wherein the tether has at the proximal end a proximal interference member secured to the distal end of the of the pushing member; the proximal interference member restricting longitudinal movement in a distal direction of the tether through the inner channel of the proximal section of the braided structure.

15. The method in accordance with claim 14, wherein the non-insulated detachment zone is at an interface between the proximal interference member and the distal end of the pushing member.

16. The method in accordance with claim 11, further comprising the step of releasing the braided implantable device from the pushing member via a mechanical detachment system.

17. The method in accordance with claim 16, wherein the tether has at the proximal end a detachment member having an opening defined therein; and the mechanical detachment system comprises:
a securement wire having a distal end with a loop projecting from the distal end of the pushing member; and
a release wire transitionable between a secured state with the release wire passing through the loop of the securement wire and the opening of the detachment member securing together the braided implantable device and the pushing member and a released state with the release wire freed from the loop of the securement wire and the opening of the detachment member releasing the braided implantable device from the pushing member.

18. A method for manufacture of an implantable endovascular embolization device, the method comprising the steps of:
providing a braided structure having a proximal edge;
cinching a proximal securement marker about the braided structure including the proximal edge creating a proximal section defining longitudinally therethrough an inner channel having an inner diameter smaller relative to that of an interior space disposed distally thereof;
providing a tether no portion of which is rotatable relative to itself;
advancing the tether through the inner channel of the proximal section of the braided structure with the distal end disposed distally of the proximal section of the braided structure in the interior space of the braided structure and the proximal end of the tether disposed proximally of the proximal section of the braided structure; sufficient radial clearance space being provided of the tether within the inner channel to allow the braided structure unrestricted 360 degrees freely rotatable movement relative to the pushing member;
attaching to the distal end of the tether within the interior space of the braided structure a distal interference member and attaching a distal end of a pushing member either directly or indirectly to the proximal end of the tether.

19. The method in accordance with claim 18, wherein the proximal end of the tether is indirectly permanently attached to the distal end of the pushing member via a proximal interference member; wherein the proximal interference member restricts passage in a distal direction in the inner channel of the proximal section of the braided structure.

20. The method in accordance with claim 18, wherein the proximal end of the tether is indirectly releasably mechanically attached to the distal end of the pushing member via a detachment member; wherein the detachment member restricts passage in a distal direction in the inner channel of the proximal section of the braided structure.
